(19) United States
(12) Patent Application Publication
(10) Pub. No.: US 2021/0259940 A1
(43) Pub. Date: Aug. 26, 2021

(54) Ultraviolet Indicators, Formulations, and Suncare Kits Comprising the Same

(71) Applicant: SunFly Brands, Inc., Seattle, WA (US)

(72) Inventors: Muhammad Khaled Arafeh, Seattle, WA (US); Michael J. Croix, Seattle, WA (US); Neil R. Branda, Seattle, WA (US); Barry Van Gemert, Seattle, WA (US); Leslie M. Watts, Seattle, WA (US)

(21) Appl. No.: 17/255,068

(22) PCT Filed: Jun. 26, 2019
(86) PCT No.: PCT/US2019/039355

§ 371 Date: Dec. 22, 2020

Related U.S. Application Data
(60) Provisional application No. 62/690,235, filed on Jun. 26, 2018.

(51) Int. Cl. A61K 8/49; A61K 8/11; A61K 8/02; A61K 8/891; A61K 8/29; A61K 8/27; A61K 8/35; A61K 8/37; A61K 8/25; A61Q 17/04

(52) U.S. Cl. CPC A61K 8/4986; A61K 2800/884; A61K 8/11; A61K 8/0283; A61K 8/891; A61K 8/29; A61K 8/27; A61K 8/35; A61K 8/37; A61K 8/25; A61Q 17/04; A61K 2800/412; A61K 2800/438; A61K 2800/56; A61K 2800/654; A61K 8/492

(57) ABSTRACT

Described herein is UV-responsive ink capable of informing the user of UV exposure in real time, and a sun care kit incorporating the same.

### CROSS REFERENCE TO RELATED APPLICATION

This application claims the benefit under 35 U.S.C. § 119(e) to U.S. Provisional Application 62/690,235 filed Jun. 26, 2019, which application is incorporated by reference herein in its entirety.

### BACKGROUND

### Technical Field

This disclosure is related to ultraviolet (UV) indictors that inform users of their exposure to ultraviolet (UV) radiation, which may be used in combination with UV-protective agents.

### Description of the Related Art

Overexposure to UV radiation is a health hazard. Skin is particularly susceptible to photodamage caused by excess exposure to UV radiation. The harmful effects of UV radiation such as in sunlight can be acute or chronic. The acute effects include erythema (e.g., redness associated with sunburns), edema, blistering and sloughing. Long-term consequences of photodamage can lead to premature aging (photo-aging), hyperpigmentation, and cancers in the skin. Eyes can also be damaged by excessive UV exposure. Acute effects arising from short-term exposure include photo-conjunctivitis; whereas long-term exposure may lead to cataracts.

Sunscreens are conventionally used to protect skin from the harmful UV radiation. Sunscreens typically contain photo-protective agents that, once applied to skin, attenuate the level of UV radiation reaching the skin. For example, mineral-based sunscreens such as titanium dioxide (TiO₂) and zinc oxide (ZnO) are generally UV blockers that reflect and scatter the UV radiation, thus forming a barrier between the UV radiation and the skin. In an alternative form of photo-protection, sunscreens may absorb UV radiation through photosensitive chemical agents. These chemical agents are generally organic compounds that absorb the photon energy of the UV radiation and are excited to a higher energy state. The organic compounds will return to a lower energy ground state with concomitant loss of energy as heat.

The effectiveness of currently available sunscreen products is typically communicated to the user through Sun Protection Factor (SPF) values. However, SPF is an imperfect metric because it is a relative measure of the amount of sunburn protection provided by sunscreens (relative to unprotected skin). Thus, a person wearing a sunscreen of SPF 15 should be able to stay in the sun 15 times longer than that same person not wearing sunscreen without getting a sunburn.

Generally, UVB radiation is responsible for sunburns, whereas UVA radiation is primarily responsible for photo-aging and hyperpigmentation of the skin. Both UVB and UVA can cause skin cancer. “Broad-spectrum” designations of sunscreens in the United States and Canada indicate both UVB and UVA protection, whereas other countries use their own designations to indicate UVA protection. For example, UVAPF stands for UVA Protection Factor, and is used in Europe to indicate UVA protection. PPD stands for Persistent Pigment Darkening, and is used in Europe and Asia to indicate UVA protection. Finally, PA stands for Protection Grade of UVA and is used in certain Asian countries to indicate UVA protection as well.

The problem with all of these measures of protection is that they cannot take into account real-life variables such as the intensity of the sun's radiation, how much a sunscreen user is swimming, sweating or toweling-off or even how much sunscreen they applied in the first place. Multiple studies show that people only apply ¼-½ of the sunscreen they should and don't reapply often enough to achieve the rated UV protection of their sunscreen. See e.g., Azurdia R M, et al. Sunscreen Application by Photosensitive Patients is Inadequate for Protection. British Journal of Dermatology. 1999 February; 140(2):255-8; Bimczok R, et al. Influence of Applied Quantity of Sunscreen Products on the Sun Protection Factor—A Multicenter Study Organized by the DGK Task Force Sun Protection. Skin Pharmacol Physiol 2007; 20:57-64; 3. Diffey B. Sunscreen Isn't Enough Journal of Photochemistry and Biology 2001 Nov. 15; 64(2-3): 105-8; 4; Neale R, et al. Application Patterns Among Participants Randomized to Daily Sunscreen Use in a Skin Cancer Prevention Trial. Arch Dermatol. 2002; 138(10):1319-1325. doi:10.1001/archderm.138.10.1319.

Users who wear sunscreen generally have no reliable way of telling whether they have applied enough sunscreen, or when to reapply. Thus, there is a need in the art to accurately inform those that are exposed to UV radiation (including sunscreen users) of the type and intensity of their UV exposure in real time and based upon current conditions, thus enabling them to apply or reapply sunscreen, or employ other UV reducing behaviors such as seeking shade.

### BRIEF SUMMARY

Provided herein are UV indicators that accurately inform users in real time of their UV exposure. Also provided are UV-responsive dermatological formulations and sun-care kits incorporating the same.

Thus, one embodiment provides a UV-responsive ink formulation comprising: a dermatologically acceptable liquid carrier; and one or more photochromic dyes.

In various embodiments, the one or more photochromic dyes of the UV-responsive ink formulation are selectively responsive to UVB radiation (290 nm-320 nm) over UVA radiation (320-400 nm).

In other embodiments, the one or more photochromic dyes of the UV-responsive ink formulation are sensitive to one or more UV intensities corresponding to UV Indices.

In various embodiments, the one or more photochromic dyes of the UV-responsive ink formulation may be a compound of spirooxazine, diarylethene, spiropyran, chromene, naphthopyran or azobenzene.

In various embodiments, the photochromic dye is encapsulated in a plurality of microcapsules, each microcapsule comprising a shell enclosing a cavity, in which the photochromic dye is suspended in a liquid solvent. In more specific embodiments, the microcapsules have diameters in the range of 1-20 μm

In other embodiments, the photochromic dye is incorporated in a plurality of solid microparticles. In more specific embodiments, each solid microparticle comprises a polymer matrix, and wherein the photochromic dye is physically embedded in the polymer matrix or chemically bonded to the polymer matrix. In certain embodiments, the microparticles have diameters in the range of 0.1-20 μm.

In yet other embodiments, the photochromic dye is conjugated to one or more oligomers having weight-average molecular weight of less than 5000.

In various embodiments, the one or more photochromic dyes of the UV-responsive ink formulation can be represented by Formula (I) or Formula (II), as defined herein.

Also provided herein is a two-part sun care kit comprising: a first compartment containing a sunscreen composition; and a second compartment containing a UV-responsive ink formulation according to the various embodiments disclosed herein.

A further embodiment provides a method for managing direct UV-exposure to mammalian skin in need thereof, the method comprising: forming an imprint of photochromic dye on the mammalian skin by applying a UV-responsive ink containing one or more photochromic dye(s) to the mammalian skin and allowing the UV-responsive ink to dry, and applying a sunscreen composition on the mammalian skin and over the thin film of photochromic dye, whereby the imprint shows a first color.

In additional embodiment, the method further comprises re-applying a sunscreen composition when the imprint changes color from the first color to a second color.

Yet another embodiment provides a multi-layer sticker comprising: a substrate; a dye layer overlying the substrate, wherein the dye layer comprises a broad-spectrum photochromic dye; a filter layer overlying the dye layer, wherein the filter layer comprises one or more UV filters selectively absorbing certain UV wavelength ranges.

In various embodiments, the UV filter of the multi-layer sticker selectively absorbs UVB (290 nm-320 nm), whereby only UVA radiation can reach the dye layer.

In other embodiments, the filter layer comprises a UVB filter (absorbing 290-320 nm) and a UVA1 filter (absorbing 340-400 nm), whereby only UVA2 radiation (320-340 nm) can reach the dye layer.

In yet other embodiments, the filter layer comprises a UVB filter (absorbing 290-320 nm) and a UVA2 filter (absorbing 320-340 nm), whereby only UVA1 radiation (340 nm-400 nm) can reach the dye layer.

A further embodiment provides a method for preventing a chemical compound from transdermal delivery or minimizing systemic exposure to the chemical compound in a subject in need thereof, the method comprising:

applying a topical formulation to the subject's skin, wherein the topical formulation includes the chemical compound; a depot-forming agent; a film-forming agent and a dermatologically acceptable carrier; and

allowing the topical formulation to form a film on the subject's skin,

wherein the depot-forming agent is:

(1) a plurality of microcapsules encapsulating the chemical compound;

(2) a plurality of microparticles incorporating the chemical compound;

(3) an oligomer conjugated to the chemical compound, or

(4) the film-forming agent itself.

In various embodiments, the chemical compound is an active ingredient of sunscreen, such as oxybenzone or octinoxate.

### BRIEF DESCRIPTION OF THE SEVERAL VIEWS OF THE DRAWINGS

Other embodiments of the present invention and their advantages will be discerned after studying the Detailed Description in conjunction with the accompanying drawings in which:

FIG. 1 shows erythemally weighted spectral irradiances under certain conditions.

FIG. 2 shows the percentage of filtration or absorption of the UVB radiation as correlated to the SPF values.

FIG. 3 shows microencapsulated photochromic dyes according to one embodiment.

FIG. 4A schematically shows a UVA indicator in the form of a patch or sticker according to one embodiment.

FIG. 4B schematically shows a microencapsulated UVA indicator.

FIG. 5A shows a sticker having multiple UV indicators that are responsive to different UV Indices.

FIG. 5B shows a stamp having multiple UV indicators that are responsive to different UV Indices.

FIG. 6 shows a stamp having multiple UV indicators that are responsive to different UV wavelength ranges.

FIG. 7 shows leaching test results of photochromic dye-loaded silica powder in various solvents. (A) is the UV-VIS spectra of the photochromic dye in its respective isoforms; and (B) shows the absorption spectra of the respective supernatants following mixing the dye-loaded silica powder in seven solvents.

### DETAILED DESCRIPTION

UV radiation that reaches the Earth's surface may be classified into two types by their wavelengths, namely, UVA radiation (320-400 nm) and UVB radiation (290-320 nm). UVB radiation is primarily responsible for sunburn and other damage to skin's superficial layers. The relatively longer wavelength UVA radiation, which accounts for approximately 95% of terrestrial UV radiation, can penetrate into the deeper layers of the skin and cause photodamage, which can lead to premature aging, wrinkling and hyperpigmentation. Both UVB and UVA contribute to skin cancers. UVA radiation can be further subdivided into UVA1 (340-400 nm) and UVA2 (320 nm-340 nm), with UVA1 causing approximately 75% of UVA-related photodamage.

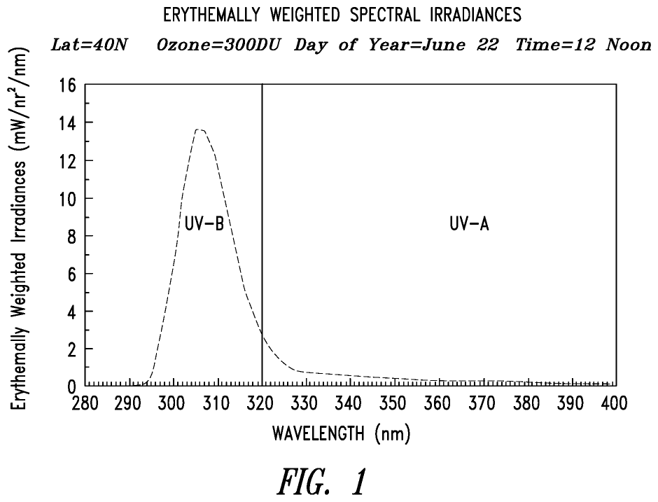

Because the skin is variably sensitive to UV radiation, a model called the Erythema Action Spectrum was adopted by the Commission Internationale de l'Eclairage (CIE) as a standard measure of the susceptibility of the Caucasian skin to sunburn (erythema) at each wavelength across the terrestrial UV range (290-400 nm). FIG. 1 shows the spectral irradiance weighted or adjusted for the Erythema Action Spectrum. As shown, although UVB accounts for approximately 5% of all UV radiation in sunlight, it is the primary cause of sunburns.

UV Index is an international standard measurement of the strength of sunburn-producing UV radiation (i.e., UVB) at a particular place and time. Using a computer model that relates the ground-level strength of solar UV radiation to forecasted stratospheric ozone concentration and forecasted cloud amounts, UV radiation can be predicted by further taking into consideration of variables such as latitude; day of the year; air pollutants, and elevation above sea level (altitude). The UV Index is then calculated based on the predicted UV radiation and weighted according to the Erythema Action Spectrum.

The UV Index is thus a predictive measure of the sun's UVB intensity at a given time and geolocation and is designed as an open-ended linear scale (0-11⁺). The United States Environmental Protection Agency (EPA) further categorizes the numerical UV Index into five levels, from Minimal (0-2.9), Moderate (3-5.9), High (6.0-7.9), Very High (8.0-10.9) to Extreme (11 and higher). The EPA guidelines correlate the levels of UV Index to appropriate protective actions, such as applying sunscreen, wearing protecting clothing and sunglasses, seeking shelter, etc.

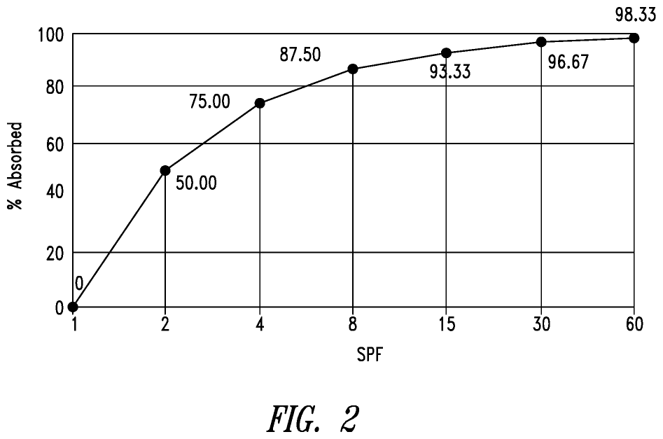

Sunscreen protects the skin by filtering out or absorbing the UV radiation. The protective effect can be measured by the SPF rating of a given sunscreen. SPF is a relative value that compares the minimal erythemal dose (MED)—the time it takes for reddening or sunburn to start—in skin protected with sunscreen and the MED in unprotected skin of persons with the same skin type. SPF can also be correlated to the relative amount of UVB protection a sunscreen provides. FIG. 2 shows the percentage of filtration or absorption of UVB radiation as correlated to the SPF values. Therefore, SPF is a relative measure of UVB protection—relative to skin type and MED, as well as percent filtration or absorption of UVB—whereas the UV index is an absolute measure of UVB intensity. Therefore, the same SPF rated sunscreen will offer different amounts of UVB protection at different UV indexes.

A full spectrum or broadband sunscreen can filter out both UVB and UVA radiation. However, the SPF rating only measures protection against UVB radiation, without accounting for protection (if any) against UVA radiation. Given the many ways to calculate UVA protection throughout the world (as described in the Description of the Related Art section above) and the relativity of UVB protection provided by SPF ratings at different UV indexes, provided herein are UV indicators that can indicate to the user the type and intensity of UV exposure reaching their skin in nearly real time.

### UV Indicators

Because skin responds in various ways depending on the wavelength and strength of the UV radiation, surrounding environment such as reflective surfaces, and skin type, UV indicators that can accurately inform users of the intensity and kind of UV exposure reaching their skin are important tools to practice proper sun protective behavior. Thus, described herein are UV indicators based on one or more photochromic dyes that respond to UV radiation of certain wavelength and/or strength by changing color. Advantageously, the UV indicators may be selectively calibrated or tuned to respond to subclasses of UV radiation, including UVB, UVA1 and UVA2 radiation, or blended to show the relative contributions of different UV wavelengths.

### Photochromic Dyes

Photochromic dyes are chemical compounds capable of changing color upon photon irradiation. The color change is a result of structural changes induced by the absorption of photon energy by the dye compound, whereby a first isomer of the dye compound associated with a first color restructures to a second isomer associated with a second color that is different from the first color. The photo-induced color change of a photochromic dye is at least partially reversible. In a reversible reaction, the second isomer of the dye compound is capable of reverting back to the first isomer under conditions such as when the initial photon irradiation ceases or when the second isomer absorbs different photon energy.

Depending on their molecular frameworks, photochromic dyes may be responsive to a wide range of photon energy. As used herein, “responsive to” “reactive to” or “activated by” photon energy refer interchangeably to the ability of a photochromic dye to absorb certain photon energy and undergo structural changes accompanied by color changes. As further described in more detail below, dyes may be tuned to respond to different ranges of wavelengths.

The structural changes are associated with color changes from one color form to another, different color form. As used herein, a “color form” refers to any visual cues arising from the visible spectrum. In some embodiments, the color form seen is the complementary color of that of the wavelength(s) in the visible spectrum absorbed by a given isomer. In other embodiments, a “color form” may be colorless, i.e., it is invisible under white or full-spectrum sunlight because the isomer does not absorb any light in the visible spectrum.

In various embodiments, a photochromic dye suitable as a UV indicator is in an original, first color form when not exposed to UV irradiation (e.g., when blocked by sunscreen or shelter). It turns into a second color form upon UV irradiation (e.g., sunscreen wears off or loses effectiveness), and reverts to the first color form when the UV irradiation ceases (e.g., when the user reapplies sunscreen or seeks shelter from the sun).

In preferred embodiments, these dyes are colorless in the absence of UV irradiation and change into visible colors when exposed to UV irradiation. As the UV radiation ceases and/or as visible light/heat dominates, the color form reverts to the colorless form, a process also referred to as “fading.”

The structures of the UV-responsive dye compounds are not particularly limited so long as the molecular framework allows for the photon-induced structural isomerization. The process should be at least partially reversible when the UV irradiation ceases. Typically, the structural isomerization may involve reversible ring-closing and ring-opening reactions; cis and trans isomerization, hydrogen, electron and functional-group transfers within the molecular framework.

### Photochromic Dyes as Broadband or Full Spectrum UV Indicators

UV-responsive photochromic dyes that are reactive to both UVA and UVB radiation are also called full spectrum or broadband indicators. Because up to 98% of UV irradiation reaching the earth is UVA, UVA can be 30 to 50 times more prevalent than UVB. Given the large discrepancy between the relative amounts of UVA and UVB in sunlight, a broadband UV indicator that does not take into account the relative proportions of UVA versus UVB in sunlight will make a poor indicator. An indicator that isn't more reactive to UVB than UVA would likely produce false positives and potentially false negatives as well, depending on its UVB reactivity. Thus, photochromic dyes selectively responsive to UVB while having low reactivity to UVA are preferred broadband indicators. In various embodiments, “selective” refers to a photochromic dye being at least 10 times more reactive to UVB radiation than to UVA radiation. In preferred embodiments, the photochromic dyes disclosed herein are at least 20 time, or at least 30 times, or at least 40 times, or at least 50 times more reactive to UVB radiation than to UVA radiation.

Examples of UV-responsive photochromic dyes that are reactive to both UVA and UVB include, without limitation, spirooxazines, diarylethenes, chromenes, spiropyrans, azobenzenes, fulgides, di-hydropyrenes, donor-acceptor Stenhouse adducts, and the like. Suitable photochromic dyes include those disclosed in, for example, U.S. Published Patent Application No. 2002/0022008 and US2016/0089316A1, U.S. Pat. No. 4,816,584, which references are incorporated herein by reference in their entireties.

In more specific embodiments, the photochromic dye is a spiro(indoline)benzoxazine compound. Thus, in certain embodiments, the photochromic dye is represented by Formula (I):

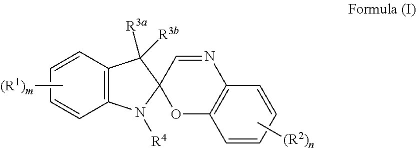

wherein,

m is 0, 1, 2, 3 or 4;

n is 0, 1, 2, 3 or 4;

R¹ at each occurrence is the same or different and independently alkyl, halo, alkoxy, haloalkyl, cyano, nitro, amino, alkenyl, alkynyl, aryl, aralkyl, heteroaryl, cycloalkyl, heterocyclyl, heteroarylalkyl, cycloalkylalkyl, or heterocyclylalkyl, or two adjacent R¹ together with the carbons to which they are attached form a carbocyclic ring;

R² at each occurrence is the same or different and independently alkyl, halo, alkoxy, haloalkyl, cyano, nitro, amino, alkenyl, alkynyl, aryl, aralkyl, heteroaryl, cycloalkyl, heterocyclyl, heteroarylalkyl, cycloalkylalkyl, or heterocyclylalkyl; or two adjacent R² together with the carbons to which they are attached form a carbocyclic ring;

each R^{3a} and R^{3b} is independently hydrogen, alkyl, or haloalkyl; and

R⁴ is alkyl, haloalkyl, aryl, heteroaryl, aralkyl, heteroarylalkyl.

As shown in Scheme 1, the spiro(indoline)benzoxazine (SIB) dyes in accordance with various embodiments selectively absorb UVB rays and undergo ring-opening isomerization to produce a charge-separated, ring-open form represented by Formula (Ia).

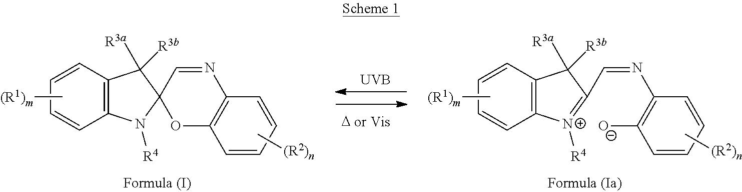

When the UVB radiation ceases, the reverse isomerization from Formula (Ia) to Formula (I) may spontaneously take place thermally or under visible light (Vis) irradiation.

The SIB dyes are temperature-dependent in that the rates of thermal fading (reverse reaction) vary depending on ambient temperatures. The color form may become unstable due to temperature change.

In a specific embodiment, the SIB dye has the following the following isomeric structures:

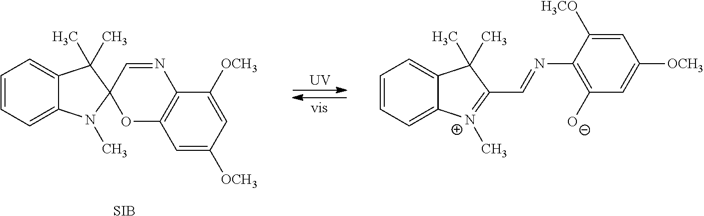 [SMILES: COc1cc(OC)c2c(c1)OC1(C=N2)N(C)c2ccccc2C1(C)C ; "UV" ; "vis" ; COc1cc([O-])c(/N=C/C2=[N+](C)c3ccccc3C2(C)C)c(OC)c1 ; "SIB"]

In other embodiments, the UV indicators may comprise temperature-independent dyes. Because these dyes' responsiveness is not affected by temperature changes, they are suitable as all season UV-indicators. As shown in Scheme 2, temperature-independent dyes of an original, color form 1 (e.g., colorless) are reactive to UV radiation (hν₁) by changing to color form 2. This form is stable in the dark. The reverse isomerization occurs when the color form 2 absorbs light of a different wavelength (hν₂, e.g., visible light).

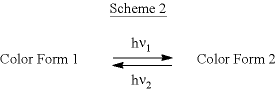

Certain diarylethene dyes are temperature-independent dyes that revert to the original color form only under visible light radiation. They are generally thermally stable in both their forms below 60° C. In certain specific embodiments, the photochromic dyes are dithienylethene compounds (DTE) represented by Formula (II):

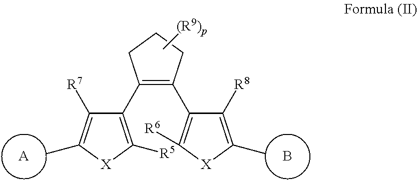

wherein,

p is 1, 2, 3, 4, 5, or 6;

A and B are the same or different and independently hydrogen, alkyl, halo, alkoxy, haloalkyl, a carbonyl-containing functional group (carboxylic acid, amide, ester, ketone, aldehyde), cyano, nitro, amino, alkenyl, alkynyl, aryl, aralkyl, heteroaryl, cycloalkyl, heterocyclyl, heteroarylalkyl, cycloalkylalkyl, or heterocyclylalkyl;

R⁵ and R⁶ are the same or different and independently alkyl, aryl or heteroaryl;

R⁷ and R⁸ are the same or different and independently hydrogen, alkyl; or

R⁷ or R⁸ connects to a respective carbon of A or B to form a benzene ring;

R⁹ is hydrogen or halogen; and

X is S or O.

As shown in Scheme 3, the dithienylethene dyes in accordance with the various embodiments undergo ring-closure isomerization under UV radiation and the reverse isomerization occurs under visible light.

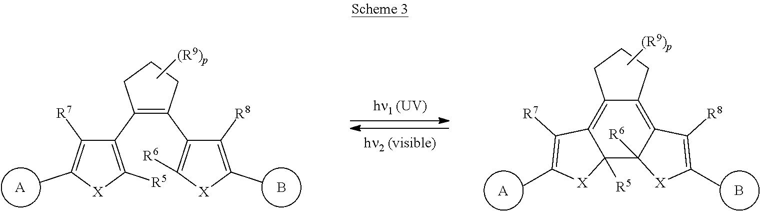

In various embodiments, the functional groups such as R⁵, R⁶, R⁷, X and A and B can be calibrated or tuned to provide dyes of certain colors or of different sensitivities to specific UV wavelength or strength. For instance, A and B, and R⁵ and R⁶ may determine the colors of the colored forms 1 and 2 as well as determine how far into the UVA (vs. UVB) Color Form 1 (e.g., colorless) absorbs. Y is preferably sulfur (S).

In a specific embodiment, the DTE dye has the following isomeric structures:

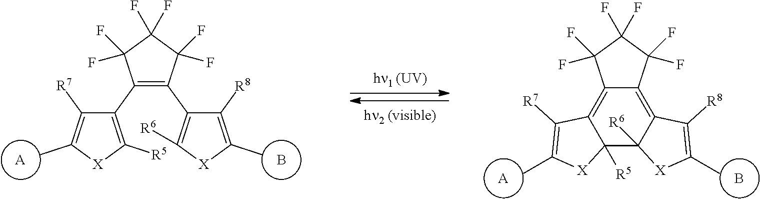

In another specific embodiment, the DTE dye has the following isomeric structures:

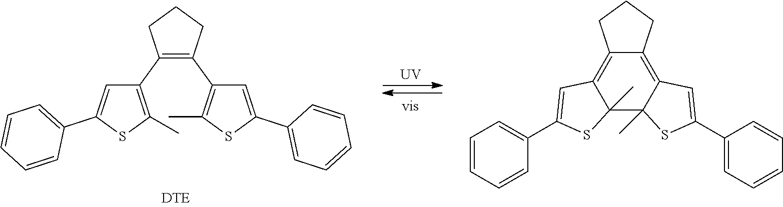

As used herein, “aryl” refers to aromatic monocyclic or multi-cyclic hydrocarbon ring system, when unsubstituted, consisting only of hydrogen and carbon and containing from 6 to 19 carbon atoms, preferably 6 to 10 carbon atoms, where the ring system may be partially or fully saturated. Aryl groups include, but are not limited to groups such as fluorenyl, phenyl and naphthyl. The aryl moiety may be substituted by one or more substituents, as defined herein.

“Alkyl” refers to a straight or branched hydrocarbon chain radical, when unsubstituted, consisting solely of carbon and hydrogen atoms, containing no unsaturation, having from one to twenty carbon atoms, preferably one to twelve, and preferably one to eight carbon atoms or one to six carbon atoms. Examples include methyl, ethyl, n-propyl, 1-methylethyl (iso-propyl), n-butyl, n-pentyl, 1,1-dimethylethyl (t-butyl), and the like. The alkyl moiety may be substituted by one or more substituents, as defined herein.

“Alkenyl” refers to a straight or branched hydrocarbon chain radical group, when unsubstituted, consisting solely of carbon and hydrogen atoms, containing at least one double bond. Alkenyl includes polyenes that may have up to 60-100 carbons, although polyenes or alkene are not limited to any number of carbons.

“Alkynyl” refers to a straight or branched hydrocarbon chain radical group, when unsubstituted, consisting solely of carbon and hydrogen atoms, containing at least one triple bond. Alkynyl may further comprise one or more double bonds.

“Cycloalkyl” refers to a stable non-aromatic monocyclic or bicyclic hydrocarbon radical, when unsubstituted, consisting solely of carbon and hydrogen atoms, having from three to fifteen carbon atoms, preferably having from three to twelve carbon atoms, and which contains no double bond in the ring structure.

“Heterocyclyl” refers to a stable 3- to 18-membered non-aromatic ring radical including, as ring atoms, at least one carbon atom and from one to five heteroatoms selected from the group consisting of nitrogen, oxygen and sulfur. For purposes of this disclosure, the heterocyclyl radical may be a monocyclic, bicyclic, tricyclic or tetracyclic ring system, which may include fused or bridged ring systems; and the nitrogen, carbon or sulfur atoms in the heterocyclyl radical may be optionally oxidized; and the nitrogen atom may be optionally quaternized; and the heterocyclyl radical may be partially or fully saturated.

“Heteroaryl” refers to a 5- to 18-membered aromatic ring radical including, as ring atoms, at least one carbon atom and from one to five heteroatoms selected from the group consisting of nitrogen, oxygen and sulfur. For purposes of this disclosure, the heteroaryl radical may be a monocyclic, bicyclic, tricyclic or tetracyclic ring system, which may include fused or bridged ring systems; and the nitrogen, carbon or sulfur atoms in the heteroaryl radical may be optionally oxidized; and the nitrogen atom may be optionally quaternized.

“Aralkyl” refers to an alkyl moiety (as defined herein) having an aryl substituent.

“Heteroarylalkyl” refers to refers to an alkyl moiety (as defined herein) having a heteroaryl substituent.

“Hetercyclylalkyl” refers to an alkyl moiety (as defined herein) having a heterocyclyl substituent.

“Substituent” refers to amino, thiol, alkyl, aryl, haloalkyl, cyano, nitro, heteroaryl, heterocyclyl and the like.

### Pigments Containing Photochromic Dye

The photochromic dyes generally should not contact skin directly or leach onto or into the skin in an amount that may elicit any adverse physiological response. Thus, to prevent the dyes from contacting the skin or leaching into the skin, the photochromic dyes are suitably incorporated into microcapsules, solid microparticles or conjugated (i.e., via covalent bonding) to oligomers to form pigments that are too large to penetrate the skin.

As used herein, “pigment” refers to dye-loaded microparticles, microcapsules, or oligomers. The dye may be incorporated by any means, including physical entrapment, chemical conjugation (e.g., by covalent bond), affinity binding, hydrophobic interaction, and the like. Typically, individual pigment has a dimension that is at least 0.1 μm, and no more than 20 μm. More typically, the pigments have dimensions in the range of 0.3 μm-10 μm, or 1-5 μm, 0.5-5 μm, 0.5-2 μm, 0.3-1.5 μm, or 0.2-1 μm. More typically, the pigments are about 1 μm. These dimensions allow the pigments to be formulated into ink that can be dispensed (e.g., through a sponge) and applied to the skin. The dye compound that remains in or is attached to the pigment is incapable of penetrating through the outer layer of the skin.

Generally, the pigment contains sufficient amount of a dye compound to allow the color change be visible to a user. In various embodiments, the pigments contain 0.01-10% (w/w) of a dye compound. In various embodiments, the pigment contains a dye compound in an amount of 0.1-10% (w/w), 0.1-5% (w/w), 0.1-2% (w/w), 0.5-1% (w/w), 0.1-1% (w/w), 0.1-3% (w/w), 1-5% (w/w), 5-10% (w/w), or 0.3-7% (w/w), and any other intermediate ranges.

1. Pigment Based on Microencapsulated Dyes

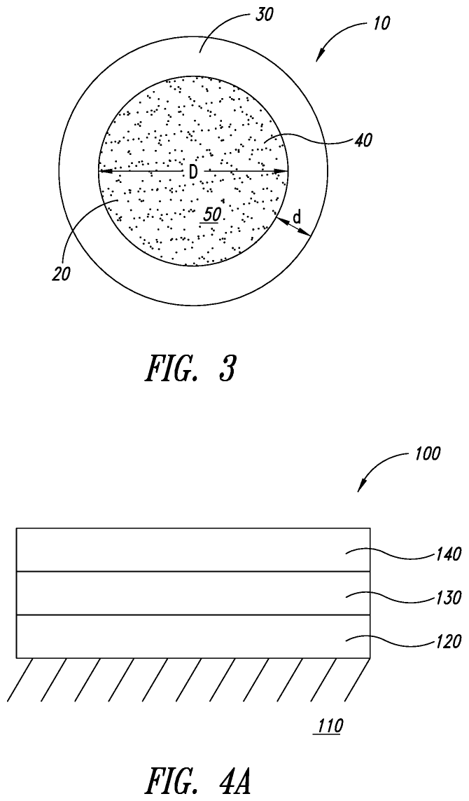

In one embodiment, the photochromic dyes (as UV indicators) may be encapsulated in microcapsules. The microcapsules provide a micro-environment for the photochromic change undertaken by the dye compounds. As schematically shown in FIG. 3, a microcapsule (10) has a cavity (20) enclosed by a spherical or near spherical shell (30). Contained within the cavity (20) is photochromic dye (40) dissolved in a solvent (50). Typically, the sizes or the diameters (D+2d) of the microcapsules are in the range of 1-20 μm, more suitably in the range of 1-10 μm. In various embodiments, the thickness of the shell (d) is less than 10%, or less than 20%, or less than 30%, or less than 40% of the diameter (D+2d) of the microcapsule. In various embodiments, the thickness of the shell (d) is in the range of 200 nm to 5 μm.

Suitable solvents are typically non-toxic, non-flammable solvents that are immiscible with water and have a low vapor pressure at 100° C. and a boiling point higher than 160° C. For photochromic dyes that are in charge-separated form (e.g., Formula (Ia)) when in colored forms, polar solvents may be employed to stabilize the colored form. Examples of the suitable solvents include, without limitation, ethylene glycol, propylene glycol, anisole, and methyl cyclohexanone. For hydrophobic photochromic dyes such as DTE, non-polar solvents could also be suitable.

The photochromic dye solution may have a concentration of the photochromic dye in the range of 0.1-10% w/v %, or any intermediate ranges, including without limitation, 0.1-10% (w/w), 0.1-5% (w/w), 0.1-2% (w/w), 0.5-1% (w/w), 0.1-1% (w/w), 0.1-3% (w/w), 1-5% (w/w), 5-10% (w/w), or 0.3-7% (w/w). Enclosing the photochromic dye solution is preferably a polymeric shell that allows transmittance of light in the UV and visible range (i.e., 290-700 nm). Because the shell is so thin, polymer materials that are either clear or opaque in their respective bulk forms can be suitable provided that they allow at least 60%, or at least 70%, or at least 80% or at least 90% of transmittance in the UV-Vis range. Thus, as used herein, a “shell” of the microcapsule is functionally transparent to UV and visible light, irrespective of the optical characteristics of the bulk material (e.g., polymer). Suitable polymer shells may be melamine-formaldehyde or urea-formaldehyde resins. Polymer shells that are required to be melamine-free or formaldehyde-free may be prepared by crosslinked gelatin.

The microencapsulation may be carried out by emulsion or double emulsion. Emulsion is a particularly suitable process by which the photochromic dye solution may be mixed with a solution of the monomers (e.g., melamine and formaldehyde) under stirring. The dye solution and the monomer solution form two phases (i.e., droplets of the monomer solutions enclosing dye solutions). The condensation and polymerization of the monomers occur at the interface of the two phases and may be initiated by known methods in the art. For instance, melamine and formaldehyde condensation is initiated by an acidic pH condition. As monomers condense into a continuous shell at the interface, the dye solution within the droplets is encapsulated.

The sizes of the microcapsules may be controlled by the microencapsulation process and the materials used. For example, in an emulsion process, the droplets of the monomer solution can be controlled by adjusting the speed of stirring.

2. Pigments Based on Dye-Incorporated Solid Microparticles

In some embodiments, the photochromic dyes are incorporated into solid microparticles. Similar to the microcapsules, the solid microparticles have dimensions (e.g., at least 0.1 μm in diameter) that prevent penetration into the skin. As used herein, “incorporated” refers to a compound (e.g., a dye compound) being physically or chemically integrated into the microparticles. Physical integration does not involve formation of chemical bonds and may include entrapment, entanglement, hydrophobic interaction, and the like. Chemical integration (also referred to as “conjugation”) involves the formation of at least one covalent bond or hydrogen bond.

Typically, the solid microparticles are formed of one or more polymeric materials. The long molecular chains of the polymer material are entangled or crosslinked, thereby creating interstitial space in which the dye compound can be entrapped. The polymer materials of microparticle thus act as a host or matrix (also referred to as “polymer matrix”) for the dye compounds. The polymer materials may be organic based, in which the molecular chains comprise C—C bonds, or C—O bonds; or inorganic-based, in which the molecular chains comprise Si—O bonds.

In some embodiments, the polymer is a low-Tg polymer (e.g., having a glass transition temperature of below 25° C., such as 0-20° C.). Low-Tg polymers have certain flexibility or “softness” at the microscopic level, enabling them to function as a solid solvent that facilitates the structural changes (isomerization). An exemplary class of low-Tg polymers include polysiloxanes. These silicone-based elastomers are chemically versatile and tend to have low-temperature flexibility in addition to high-temperature stability. An example of suitable polysiloxanes is poly(dimethylsiloxane) (PDMS). In various embodiments, the molecular weight of the PDMS is suitably in the range of 700-10000 Daltons. Unless specified otherwise, the molecular weight discussed herein refers to weight average MW. In other embodiments, polymer materials with relatively high Tg can be modified to reduce its Tg to a range that facilitates the dye structural changes. In some embodiments, a polymer may be modified by appending or conjugating an oligomer adduct (i.e., an oligomer covalently conjugated to a dye). These modified polymers may also be referred to as “low-Tg” polymers.

Examples of the modifiable polymer polymers such as polyacrylate, poly(vinyl acetate), poly(vinylalcohol), poly(vinylchloride), poly(vinlylidene chloride), polyurethanes, polycarbonates, poly(ethylene-terephthalate), polystyrene, copoly(styrene-methylmethacrylate), copoly(styrene-acrylateonitrile), poly(vinylbutryal), and homopolymers and copolymers of diacylidene pentaerythritol, particularly copolymers with polyol(allylcarbonate) monomers, e.g. diethylene glycol bis(allyl carbonate), and acrylate monomers.

Examples of the oligomer adduct includes a polysiloxane oligomer (e.g., PDMS). The molecular weight of the polysiloxane oligomer may be suitably less than 5000. In some embodiment, the polysiloxane oligomers (including PDMS) have molecular weight in the range of 750-2000. The dye compounds may be first functionalized to create one or more reactive groups capable of conjugating with the oligomer. Optionally, linkers such as polyethylene oxide may link the dye to the oligomer. More detailed description of modifying rigid polymers to provide low-Tg polymers may be found, for example, in U.S. Pat. Nos. 7,807,075, 8,865,029, 9,250,356, 9,250,356, and 9,217,812, which patents are incorporated herein by reference in their entireties.

The polymer material can form solid particles by known methods in the art, including without limitation, polymeric nanoparticles or microparticles, self-emulsifying delivery systems, liposomes, microemulsions, micellar solutions and solid lipid nanoparticles (SLN) formation.

The polymer material (e.g., PDMS) may be first pre-made into microparticles. Thereafter, the dye may be entrapped within the polymer matrix of by imbibing or swelling the pre-made microparticles in a dye-containing solution,

Alternatively, the polymer is first combined with a dye-containing solution and made into solid microparticles while simultaneously entrapping the dye during the microparticle formation. Suitable polymers include polysiloxanes.

In yet another alternative embodiment, monomers of the polymer matrix may be polymerized in the presence of a dye solution, thereby entrapping the dye during the polymerization. The dye-loaded polymer can be milled or otherwise resized into desired size ranges.

In a specific embodiment, a dye compound is entrapped in silica. Dye-loaded silica may be formed by a sol-gel process, whereby a silica precursor material (e.g., tetraethylorthosilicate) is hydrolyzed under neutral or acidic condition to produce hydrated silica (a gel). The hydrated silica has a complex molecular network or lattice that can uptake the dye. Following solvent removal (e.g., by evaporation), dye-loaded silica glass is formed, which can then be broken down (e.g., by milling) into silica powder of desired sizes.

In other embodiments, the photochromic dyes may be modified to contain oligomers which entangle with the polymer chains to prevent the dyes from leaching or escaping. Alternatively, the polymer carrier may be selected to contain hydrophobic interiors, in which the typically hydrophobic dyes can be contained due to hydrophobic and hydrophilic interactions.

In yet other embodiments, the photochromic dyes may be chemically modified by attaching a polymerizable moiety such as an acrylate or methacrylate. The modified dye can be co-polymerized with monomers to create polymerized dye. For instance, an acrylate moiety can be attached to the A or B ring of a DTE dye. The resulting dye-conjugated polymer may be ground up into solid particles of the appropriate sizes or formed into micron and submicron microparticles by, and not limited to, any of the known methods described above.

3. Pigments Based on a Dye Conjugated to Oligomers

Oligomers typically have molecular weights in the range of thousands, and may even be less than 1000. They nevertheless have long molecular chains that can entangle and form a bulky mass having at least 0.1 μm in dimension, which will not penetrate the skin. Thus, pigments of a dye conjugated to one or more oligomers may also be used to prevent transdermal delivery of the dye compound.

Suitable oligomers include polysiloxanes, including for example, PDMS. Typically, the oligomers have a weight average molecular weight of less than 5000, or more preferably, less than 4000, or less than 3000, or less than 2000, or less than 1000.

A dye compound may be modified to create one or more functional groups that are reactive to the oligomer, either directly or through a linker. The resulting dye-oligomer conjugates have a dimension of at least 0.1 μm.

In some embodiments, the dye-oligomer conjugate are the same as the dye-oligomer adducts described in U.S. Pat. Nos. 7,807,075, 8,865,029, 9,250,356, 9,250,356, and 9,217,812.

### Depot-forming Agents

It should be noted that the various embodiments of the pigment formation discussed herein are also applicable to other chemicals that are desired to remain on the skin with minimal or no transdermal penetration. The microcapsules, microparticles, and oligomers are referred to as “depot-forming agents.” These agents are capable of preventing the chemical compound to which they are coupled from transdermal delivery. For instance, some of the active ingredients (e.g. oxybenzone) in sunscreens are known to be delivered transdermally, causing high systemic absorption and potentially negative physiological effects. These active ingredients are suitable to be coupled to the various types of depot-forming agents, including being encapsulated in microcapsules, incorporated into solid microparticles, conjugated to an oligomer in the same manner as described herein in connection with the pigments. Alternatively, the oligomer conjugates can also be used as a mode of local application of pharmaceutical drugs for treating dermatological conditions while maintaining low systemic exposure to the drugs.

Thus, a further embodiment provides a method for preventing a chemical compound from transdermal delivery or minimizing systemic exposure to the chemical compound in a subject in need thereof, the method comprising:

applying a topical formulation to the subject's skin, wherein the topical formulation includes the chemical compound; a depot-forming agent; a film-forming agent and a dermatologically acceptable carrier; and

allowing the topical formulation to form a film on the subject's skin,

wherein the depot-forming agent is:

(1) a plurality of microcapsules encapsulating the chemical compound;

(2) a plurality of microparticles incorporating the chemical compound;

(3) an oligomer conjugated to the chemical compound; or

(4) the film-forming agent itself.

In various embodiments, the chemical compound may be one or more photochromic dye.

In other embodiments, the chemical compound may be one or more active ingredients in sunscreens, as described herein. In specific embodiments, the active ingredient is oxybenzone or octinoxate.

In various embodiments, the depot-forming agent comprises polysiloxanes (e.g., PDMS), or silica, or PDMS oligomer.

In various embodiments, the film-forming agent could either itself act as the depot-forming agent or facilitate the film-forming process of the other types of depot-forming agents. Examples of the film-forming agent include Shellac, nitrocellulose, hydroxymethylcellulose, hydroxyethylcellulose and zein.

### UVB Indicators

As discussed herein, there are benefits for indicators that are selectively responsive to UVB radiation (290-320 nm), i.e., more reactive to UVB radiation as compared to UVA radiation. Photochromic dyes that are selectively responsive to UVB radiation thus have the benefit of not being overwhelmed by the abundant UVA radiation in sunlight. The selectivity enables these dyes to act as UVB-indicators that can accurately inform the user of UVB exposure, which is primarily responsible for sunburns and of which SPF is a factor of. Advantageously, UVB indicators can be calibrated or tuned to respond to specific strength of the UV radiation (e.g., a particular UV Index) and indicate to the user their absolute UVB exposure irrespective of their sunscreen's SPF, how intense the sun is, how much sunscreen they applied, or other variable conditions. As discussed herein, the UV Index is a linear scale directly proportional to the intensity of UVB radiation that causes sunburn. Thus, various embodiments are directed to UVB indicators in which a discernable color change can be affected when the UV Index is 3 or above. In other embodiments, more than one dye compound, each sensitive to different UV Indices may be used as UVB indicators, which can distinguish between different intensities of the UVB radiation.

Certain spiro(indoline)benzoxazines encompassed by Formula (I) are selectively reactive to UVB radiation, with little or no absorption of UVA radiation. In a specific embodiment, the UVB indicator comprises a dye compound of Formula (I) in respective ring-closed and ring-opened forms shown below:

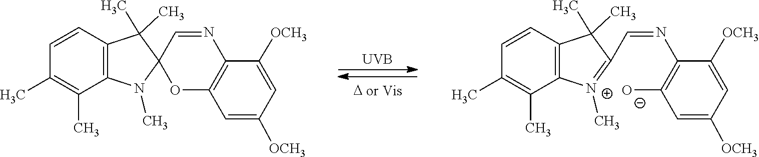 [SMILES: COc1cc(OC)c2c(c1)OC1(C=N2)N(C)c2c(ccc(C)c2C)C1(C)C ; "UVB" ; COc1cc([O-])c(/N=C\C2=[N+](C)c3c(ccc(C)c3C)C2(C)C)c(OC)c1]

Certain dithienylethene dyes are selective UVB indicators. In specific embodiments, compounds of Formula (II) are UVB selective when A and B are alkyl, R⁵ and R⁶ are alkyl, R⁷ and R⁸ are hydrogen and R⁹ is fluorine.

As with many photochromic dyes, the UVB indicator can be microencapsulated. In one embodiment, the UVB indicator is a spiro(indoline)benzoxazine and the solvent polycaprolactone diol. In another embodiment the compound is a dithienylethene and the solvent a dibasic ester solvent under the Tradename Rhodiasolve® RPDE obtainable from Solvay. In other embodiments, the UVB-indicator may be dissolved into a polymer carrier or co-polymerized before being formed into solid microparticles between 100 nm and 10 μm in size.

Another method for creating a selective UVB indicator is to combine a UVA filter with a broadband UV indicator (i.e., a photochromic dye that is reactive to both UVA and UVB radiation).

The UV indicators may be any broadband photochromic dye such as spirooxazines, diarylethenes, spiropyrans, azobenzenes, naphthopyrans and the like. The UVA filter can be any compound that selectively absorbs UVA radiation and releases the energy as heat when returning to the ground state.

### UVA Indicators

Photochromic dyes are generally reactive to both UVA and UVB radiation. Although UVB-only reactive photochromics, including those described herein, are known, there are far fewer photochromics currently known that are reactive to only UVA radiation. Exemplary UVA-reactive only photochromic dyes include certain dithienylethene dyes of Formula (II), wherein A and B are phenyl, R⁵ and R⁶ are alkyl, R⁷ and R⁸ are hydrogen and R⁹ is fluorine, it is UVA active.

Another method for creating a selective UVA indicator is to combine a UVB filter with a broadband UV indicator (i.e., a photochromic dye that is reactive to both UVA and UVB radiations).

The UV indicators may be any broadband photochromic dye such as spirooxazines, diarylethenes, spiropyrans, azobenzenes, naphthopyrans and the like. The UVB filter can be any compound that selectively absorbs UVB radiation and releases the energy as heat when returning to the ground state.

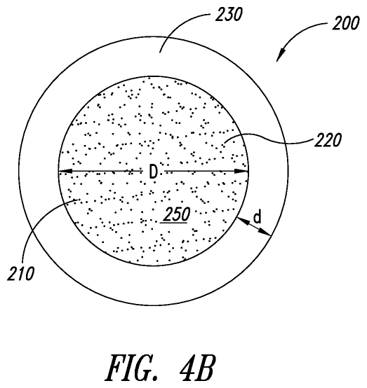

FIG. 4A schematically shows a UVA indicator according to one embodiment. As shown, the UVA indicator is in the form of a multi-layer patch or sticker (100), which can be affixed to skin (110). The patch or sticker (100) comprises a substrate (120), which is printable or coated on one side and adhesive on the skin-contacting side. A dye layer (130) of broadband photochromic dye may be coated or printed on the substrate. The dye layer (130) is then overcoated with a filter layer (140) comprising a UVB filter that is also transparent to UVA radiation. As the UVB filter absorbs the UVB radiation, the unfiltered UVA passes through filter layer (140) and activates the underlying broadband UV indicator and therefore acts as a selective UVA indicator.

In further embodiments, the filter layer (140) may comprise a UVB filter (absorbing 290-320 nm at least 80% transmittance) and a UVA2 filter (absorbing 320-340 nm at least 80% transmittance), making the underlying broadband photochromic dye a UVA1 indicator that is selectively responsive to UVA1 range of 340-400 nm.

In other embodiments, the filter layer (140) may comprise a UVB filter (absorbing 290-320 nm) and a UVA1 filter (absorbing 340-400 nm), making the underlying broadband photochromic dye a UVA2 indicator that is selectively responsive to UVA2 range of 320-340 nm.

Examples of UVB filters include certain organic agents that are known to be selectively UVB-absorbing. These filters include, without limitation, aminobenzoic acid (PABA), Uvinul T 150, Padimate O, Enzacamene, Parsol SLX, Amiloxate, cinoxate, ensulizole (phenylbenzimiazole sulfonic acid), homosalate, octocrylene, octinoxate (octyl methoxycinnamate), octisalate (octyl salicylate), 2-ethylhexyl 4-dimethylaminobenzoate, and trolamine salicylate. Other UVB filters may be inorganic substances such as TiO₂ and glass.

Examples of UVA1 filters include, without limitation, avobenzone, bisdisulizole disodium, Uvasorb HEB (UVA1 and UVB) and Helioplex (UVA1 and UVB).

Examples of UVA2 filters include, without limitation, Mexoryl XL, Meradimate, Uvinul A Plus, Mexoryl SX (UVA2 and UVB), titanium dioxide (UVA2 and UVB), octocrylene (UVA2 and UVB), oxybenzone (UVA2 and UVB), Ensulizole (UVA2 and UVB), Dioxybenzone (UVA2 and UVB) and Sulisobenzone (UVA2 and UVB).

It should be noted that a UVB and/or a UVA2 and/or a UVA1 indicator may also be in the form of the sticker, patch, wristband or other device in which a broadband UV indicator is overcoated by one or more selective filters.

In an alternative embodiment to the sticker, patch, wristband or other device, the UV indicator may be microencapsulated. FIG. 4B shows a selective UVA indicator as a microcapsule (200). Similar to FIG. 3, the microcapsule (200) comprises a cavity (210) enclosed by a shell (230). The cavity (210) contains a broadband UV indicator (220) in a solvent (250); the shell (230) comprises a UVB filter agent. Alternatively or additionally, the UVB filter could be incorporated in the cavity.

In other embodiments, UVA1 indicators may be in a microencapsulated form by incorporating broadband UV indicators in the cavity combined with UVB and UVA2 filters in the shell and/or cavity.

In yet other embodiments, UVA2 indicators may be in a microencapsulated form by incorporating broadband UV indicators in the cavity combined with UVB and UVA1 filters in the shell and/or cavity.

It should be noted that a UVB indicator may also be in the form of microcapsules in which a broadband UV indicator is incorporated in the cavity combined with a UVA filter in the shell and/or cavity.

### UV-Responsive Formulations and Wearables

In certain embodiments, the UV indicators may be incorporated in dermatological formulations, which can be applied directly to the skin. In other embodiments, the UV-indicators may be incorporated in wearable such as stickers, bracelets, jewelries, and the like. The color change provides the user with a visual signal to apply or reapply sunscreen or seek sun protective measures.

As used herein, a UV-responsive dermatological formulation (also referred to as “UV-responsive ink formulation,” or “UV-responsive ink,” or simply “ink”) comprises one or more photochromic dyes. In certain embodiments, the dyes are present in a free form in the ink. As used herein, “free form” refers to an unaltered dye compound that is not modified, conjugated to, incorporated in or having any structural relationship with the microcapsules, microparticles or oligomers, as described herein. In other embodiments, the dyes are in the form of pigments, i.e., they are encapsulated in microcapsules or incorporated in solid microparticles, or conjugated to an oligomer (such as PDMS).

Irrespective of the form in which the dye is present, the ink typically further comprises a dermatologically acceptable liquid carrier and a film-forming agent and optionally additional ingredients that facilitate the suspension and dispensing of the dye.

The dermatologically acceptable liquid carrier includes one or more solvents, which suspends the other ingredients including the free dye or pigments. The dermatologically acceptable liquid carrier is suitably non-toxic and volatile (e.g., having a boiling point of less than 100° C., more preferably less than 80° C.). Additionally, the dermatologically acceptable liquid carrier is inert and does not react with or otherwise compromise the microcapsules.

Suitable dermatologically acceptable liquid carriers include for example, alcohol such as denatured ethanol or isopropanol. Minor amounts (e.g., less than 5% or preferably less than 3%) of other organic or inorganic solvents may also be present, including for example, water, acetone, methyl isobutyl ketone. An example of suitable dermatologically acceptable liquid carrier is a solvent mixture under the tradename SD23H, which contains 97% denatured ethanol, 2% methyl isobutyl ketone and 1% acetone.

A dermatologically acceptable adhesive may also be incorporated in the UV-responsive ink to ensure that the microcapsules or solid microparticles of photochromic dyes adhere to the skin and can withstand rubbing (e.g., shear force) and moisture (water or sweat) better than the sunscreen. Additionally, the adhesive should be functionally transparent to UV and visible light. As used herein, the adhesive may also be referred to as a “film forming agent.” After the ink is applied to the skin and the solvents evaporate, the adhesive forms a thin film that conforms to the skin while immobilizing the free dye and/or pigment.

Suitable adhesives or film-forming agents include Shellac, which is a natural resin derived from secretions of lac bugs. Shellac is soluble in alcoholic solvents and dries into a clear film on the skin. Food or cosmetic grades of Shellac are commercially available. Another type of suitable film-forming agents include soluble celluloses, such as nitrocellulose, hydroxymethylcellulose, and hydroxyethylcellulose. A further type of film-forming agents include zein, a class of prolamine protein found in maize.

Other additives may also be present in the UV-responsive ink. These additives may be a thickener to assist with suspending the microcapsules or microparticles; or to facilitate adherence to the skin, or to provide water-resistance (especially to chlorinated or salt water), or to enhance stability of the UV-responsive ink (e.g., shelf-life), or to enhance texture. Examples of the additives include plasticizers, fumed silica (Cabosil®), preservatives (e.g., C₁₋₃ alkyl parabens and phenoxyenthanol), acrylates C10-30 alkyl acrylates crosspolymer, and the like.

Thus, one embodiment provides a UV-responsive ink formulation comprising:

a dermatologically acceptable liquid carrier;

a film-forming agent dissolved in the dermatologically acceptable carrier; and

a photochromic dye.

In further embodiments, the photochromic dye is encapsulated in microcapsules.

In other embodiment, the photochromic dye is incorporated in microparticles. In additional embodiments, the microparticles are PDMS microparticles. In other embodiments, the microparticles are silica particles.

Inn yet another embodiment, the photochromic dye is coupled to an oligomer. In certain specific embodiment, the oligomer is PDMS.

In various embodiments, the photochromic dye is present in the ink in an amount of 0.01-10% (w/w), or 0.1-10% (w/w), 0.1-5% (w/w), 0.1-2% (w/w), 0.5-1% (w/w), 0.1-1% (w/w), 0.1-3% (w/w), 1-5% (w/w), 5-10% (w/w), or 0.3-7% (w/w).

In certain embodiments, the UV-responsive ink may comprise a reference dye that is not photochromic. This is particularly useful when the UV indicator is colorless in Color Form 1 in the original isomer prior to converting to a second isomer (associated with Color Form 2) upon absorbing UV radiation. The reference dye serves as a visual signal of the adequate application of the UV-responsive ink. For instance, if Color Form 1 is colorless and Color Form 2 is blue, by blending a reference dye of red with the dye, it should be expected that Color Form 2 would appear purple (a blend of blue and red) in response to UV radiation. In another example, a reference dye that is not photochromic but the same color as Color Form 2 could be stamped along with (e.g. surrounding or next to) the photochromic dye to serve as a reference for color saturation and target UV intensity.

The UV-responsive ink is to be applied directly to mammalian skin. Once the UV-responsive ink contacts the skin, the volatile liquid carrier rapidly evaporates, and the free photochromic dye(s), or microcapsules or microparticles containing the photochromic dye(s), or oligomer conjugated to the photochromic dyes adhere to the skin by the strength of the adhesive, typically in the form of a thin film. The thin film has a thickness ranging from several microns up to 100 microns. The size of the ink imprint is not particularly limited other than it should be large enough to be visualized once the color change occurs.

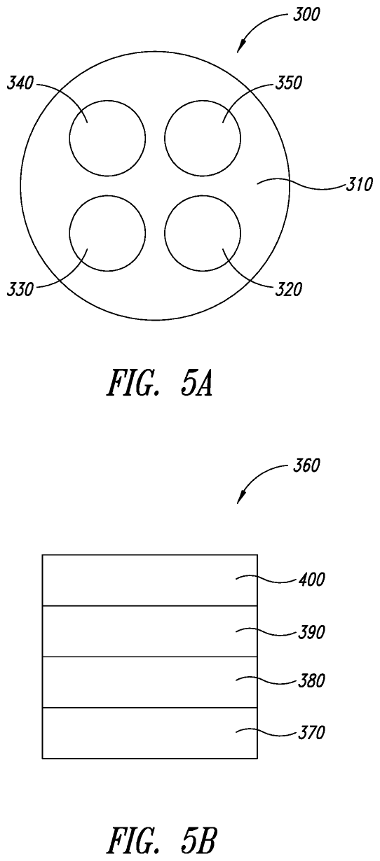

More than one UV indicators may be combined in a single stamp, sticker or device that is used to indicate the type or intensity UV radiation reaching the stamp, sticker or device. For example, multiple UV indicators that respond to UV Index 3, 6, 8 and 11 by color changes may be applied to skin at the same time, providing the user with real time information of the degree/amount of UVB exposure. FIG. 5A shows a sticker (300) having a substrate (310) printed with four different UV indicators (320, 330, 340, 350). The sensitivity of each UV indicator is tied to a UV index, for example, the index that corresponds to an EPA category (from moderate Index 3 to extreme Index 11+). The user can observe the color change of each UV indicator based on their calibrated UVB reactivities and therefore view relative increases in UVB intensity.

FIG. 5B schematically shows another embodiment wherein more than one UV indicator inks are stamped onto the skin in parallel. The stamp (360), which may be dispensed from a multi-compartment applicator, applies more than one UV indicator ink, each correlating to a different UV Index value or range. For example, UV indicators 370, 380, 390, 400 could correspond to UVB strength in the UV Indices of 3, 6, 8 and 11, respectively.

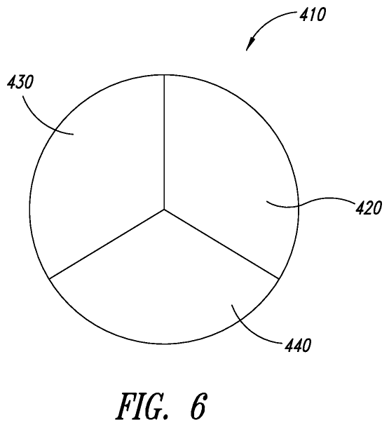

In another example, different UV indicators can indicate the different types of UV radiation reaching the indicators. FIG. 6 shows a stamp (or a sticker) (410) that has three zones of indicators (420, 430, 440), which correspond to UVB, UVA1 and UVA2 radiation, respectively. Each zone of indicator could alert the user to the type and/or intensity of UV radiation.

### Sun-Care Kit

Stand-Alone UV Indicator Dispenser

One embodiment provides a sun-care kit comprising a stand-alone dispenser that dispenses the one or more UV indicators as disclosed herein. In more specific embodiments, the UV indicators are formulated into an ink, which may be applied directly to the skin through a foam dispenser (e.g., a pre-inked foam stamp). The foam dispenser should have the necessary volume, pore size and surface chemistry to allow the ink containing the photochromic dye, in free form, or incorporated in microcapsules, microparticles or oligomers to pass through the foam and be deposited onto the skin.

A specific embodiment provides a container containing a UV-responsive ink including a dermatologically acceptable liquid carrier; an adhesive dissolved in the dermatologically acceptable carrier; and one or more photochromic dyes suspended in the dermatologically acceptable liquid carrier.

In a more specific embodiment, the one or more photochromic dyes are in free from.

In another more specific embodiment, the one or more photochromic dyes are encapsulated in microcapsules, each microcapsule comprising a shell and a photochromic dye solution encapsulated in the shell, wherein the photochromic dye solution comprises the one or more photochromic dyes dissolved in a solvent.

In a further specific embodiment the one or more photochromic dyes are incorporated in a plurality of solid microparticles, each microparticles comprising a low-Tg polymer carrier.

In yet another embodiment, the one or more photochromic dyes are conjugated to one or more oligomers (e.g., PDMS).

In various embodiments, the container is in the form of a foam dispenser, a felt pen or any applicator that can deliver the UV-responsive ink.

2. Two-Compartment Sun Care Kit

A further embodiment provides a sun care kit which combines one or more UV indicators and a sunscreen. The UV-responsive ink disclosed herein is suitably combined with a sunscreen to provide a sun care kit that informs users about their current UV exposure and helps them apply the right amount of sunscreen at the right time, or to seek other sun protection measures. As an example, a suitable two-compartment dispenser is described in WO2017/201274.

Thus, one embodiment provides a two-part sun care kit comprising:

a first compartment containing a sunscreen composition; and

a second compartment containing a UV-responsive ink including a dermatologically acceptable liquid carrier; an adhesive dissolved in the dermatologically acceptable carrier; and a plurality of microcapsules suspended in the dermatologically acceptable liquid carrier, each microcapsule comprising an shell and a photochromic dye solution encapsulated in the shell, wherein the photochromic dye solution comprises one or more photochromic dyes dissolved in a solvent.

In another embodiment, the two-part sun care kit comprises:

a first compartment containing a sunscreen composition; and

a second compartment containing a UV-responsive ink including a dermatologically acceptable liquid carrier; an adhesive dissolved in the dermatologically acceptable carrier; and one or more photochromic dyes.

In a more specific embodiment, the one or more photochromic dyes are in a free from.

In another more specific embodiment, the one or more photochromic dyes are encapsulated in microcapsules, each microcapsule comprising a shell and a photochromic dye solution encapsulated in the shell, wherein the photochromic dye solution comprises the one or more photochromic dyes dissolved in a solvent.

In a further specific embodiment, the one or more photochromic dyes are incorporated in a plurality of solid microparticles, each microparticles comprising a low-Tg polymer carrier.

In yet another embodiment, the one or more photochromic dyes are conjugated to one or more oligomers (e.g., PDMS).

As discussed herein, the low-Tg polymer carrier may be physically blended with the photochromic dye or co-polymerized with the photochromic dye.

As used herein, a “sunscreen” forms a barrier over the skin, thereby preventing certain UV radiation from reaching the skin through light reflection or scattering. In some embodiments, the sunscreens may be mineral-based and form a physical barrier. Examples include titanium oxide, zinc oxide or a mixture thereof.

In other embodiments, the sunscreens may be chemical-based that contain photo-reactive chemical agents capable of absorbing UV radiation and convert it to heat (i.e., when relaxing back to the ground state). Examples include aminobenzoic acid (PABA), avobenzone, cinoxate, dioxybenzone, ecamsule (mexoryl SX), ensulizole (phenylbenzimiazole sulfonic acid), homosalate, meradimate (menthyl anthranilate), octocrylene, octinoxate (octyl methoxycinnamate), octisalate (octyl salicylate), oxybenzone, padimate 0, sulisobenzone, trolamine salicylate, and the like.

One skilled in the art will also recognize the sunscreen composition may provide various levels of protection depending on the concentrations of the minerals or chemical agents. The level of protection afforded by the sunscreen composition can be determined by, for example, an SPF test. In the SPF test, the sunscreen composition can be applied to skin that receives a pre-determined dose of UV energy simulating sun exposure. For a product to be labeled as SPF 30 in the U.S., it must prevent sunburn until a UV dose equivalent to 30 times the minimal erythema dose (MED) is received. A skilled person in the art would appreciate that MED may be different depending on the skin type.

In addition to the sunscreen agents (including minerals or chemical agents), the sunscreen composition may contain other conventional dermatological components including oils or emollients, humectants, emulsifiers, chelating agents, preservatives, antioxidants and like. Emollients, typically present in amounts ranging from about 0.01% to 5% of the total sun block composition include, but are not limited to, fatty esters, fatty alcohols, mineral oils, polyether siloxane copolymers, and mixtures thereof. Humectants, typically present in amounts ranging from about 0.1% to about 5% by weight of the total composition include, but are not limited to, polyhydric alcohols such as glycerol, polyalkylene glycols (e.g., butylene glycol, propylene glycol, dipropylene glycol, polypropylene glycol, and polyethylene glycol) and derivatives thereof, alkylene polyols and their derivatives, sorbitol, hydroxy sorbitol, hexylene glycol, 1,3-dibutylene glycol, 1,2,6-hexanetriol, ethoxylated glycerol, propoxylated glycerol, and mixtures thereof. Emulsifiers, typically present in amounts from about 1% to about 10% by weight of the sun block composition, include, but are not limited to, stearic acid, cetyl alcohol, stearyl alcohol, steareth 2, steareth 20, acrylates/C₁₀₋₃₀ alkyl acrylate crosspolymers, and mixtures thereof. Chelating agents, typically present in amounts ranging from about 0.01% to about 2% by weight, include, but are not limited to, ethylenediamine tetraacetic acid (EDTA) and derivatives and salts thereof, dihydroxyethyl glycine, tartaric acid, and mixtures thereof. Additional antioxidants, typically present in an amount ranging from about 0.02% to about 0.5% by weight of the composition, include, but are not limited to, butylated hydroxy toluene (BHT); vitamin C and/or vitamin C derivatives, such as fatty acid esters of ascorbic acid, particularly asocorbyl palmitate; butylated hydroanisole (BHA); phenyl-α-naphthylamine; hydroquinone; propyl gallate; nordihydroquiaretic acid; vitamin E and/or derivatives of vitamin E, including tocotrienol and/or tocotrienol derivatives; calcium pantothenates; green tea extracts; mixed polyphenols; and mixtures of any of these.

The above ingredients can be formulated into a cream, a lotion, a gel, solution, an ointment, a paste or a solid stick, contained in the first compartment. The first compartment is equipped with a dispensing mechanism (e.g., pump, squeeze, or spray) consistent with the specific formulations according to known methods in the art.

The second compartment contains UV-responsive ink as described herein. The UV-responsive ink may be applied directly to the skin through a foam dispenser (e.g., a pre-inked foam stamp). As in the stand-alone dispenser, the foam dispenser should have the necessary volume, pore size and surface chemistry to allow the ink containing the photochromic dye, in a free form, or incorporated in microcapsules or microparticles, or conjugated to oligomers, to pass through the foam and be deposited onto the skin.

### Use of the Sun Care Kit

The sun care kit as described herein is suitable for managing direct UV-exposure. In particular, the UV-responsive ink is to be applied to an easily accessible and visible spot of the skin and before applying the sunscreen composition from the first compartment. The UV-responsive ink forms a thin-film imprint on the skin in a first color form (e.g., colorless) underneath the sunscreen composition. In an alternative embodiment, the UV-responsive ink includes a reference dye that is not photochromic, but provides a visual cue that the ink (along with the colorless photochromic ink) has been applied.

As the sunscreen composition wears off or otherwise loses effectiveness, the imprint changes to a second color, which signals to the user to reapply sunscreen or to avoid further sun exposure (e.g., seek shade). The second color may be a blend of the first and second color of the photochromic dye, or a blended first and second color compared to a reference dye.

Thus, one embodiment provides a method for managing direct UV-exposure to mammalian skin in need thereof, the method comprising:

forming an imprint of a photochromic dye on the mammalian skin by applying a UV-responsive ink to the mammalian skin and allowing the UV-responsive ink to dry, wherein the UV-responsive ink comprises a dermatologically acceptable liquid carrier; an adhesive dissolved in the dermatologically acceptable carrier; and a plurality of microcapsules suspended in the dermatologically acceptable liquid carrier, each microcapsule comprising a shell and the photochromic dye solution encapsulated in the shell, wherein the photochromic dye solution comprises the photochromic dye dissolved in a solvent; and

applying a sunscreen composition on the mammalian skin and over the thin film of photochromic dye, wherein the sunscreen composition comprises one or more mineral-based or chemical-based compounds, whereby the imprint shows a first color.

In an alternative embodiment, the photochromic dye is in a free form.

In yet an alternative embodiment, the photochromic dye is encapsulated in microcapsules.

In an alternative embodiment, the photochromic dye is in the form of microparticles.

In a further embodiments, the photochromic dye conjugated to one or more oligomers.

In various further embodiments, the method further comprises re-applying a sun block composition when the imprint changes color from the first color to a second color.

In other embodiments, the apparent saturation (or intensity of the color) may be adjusted depending on the specific photochromic dye compounds or their concentrations. Some dye compounds are more sensitive to a certain UV range(s), while others produce a color change that is more readily discernable to the human eye. Apparent saturation may also be adjusted by varying the concentration of photochromic compound in the UV-responsive ink and/or the thickness of the stamp. Multiple photochromic compounds can also be used to indicate different kinds and intensities of UV. These differing photochromic compounds may be combined in a single ink system, or be contained in separate ink systems that are applied simultaneously (for example as closely related parallel lines).

### EXAMPLES

### Example 1

### Free Dye in Ink

A typical ink formulation containing free DTE dye was prepared by dispersing or wetting xanthan gum with glycerin, which was stirred into an isopropanol solution of shellac and the dye. The relative amounts of the ink components are as follows:

84% isopropyl alcohol

14.0% shellac (film former)

0.5% xanthan gum

0.5% glycerin

1% dye.

### Example 2

### Forming DTE-Doped Film on Skin Mimic

A solution of Shellac (film former) (2.1 g) in Isopropanol (13 g) was treated with a mixture of acrylates C10-30 alkyl acrylates crosspolymer (texture enhancer) (75 mg) and glycerin (75 mg). The mixture was sonicated at 35 kHz for 1 h until the solution became clear.

The mixture was used as an ink solution to form a dye-loaded film on a skin mimic. A 0.5% solution of DTE in the ink was prepared by dissolving DTE dye (10 mg) in the previously prepared ink formula (2 mL). The mixture was sonicated again at 35 kHz for 1 h. The DTE-doped ink (20 μL) was deposited on the skin mimic side of previously hydrated VITRO-SKIN sample (1×1 cm), and the film was allowed to dry at room temperature for 1 h. The thickness of the resultant ink film was measured using a micrometre and was found to be 20 μm. The film became purple upon irradiation with UV light (312 nm) and returned to colorless when it was irradiated with visible light (>450 nm).

### Example 3

### Forming Pigments Using Pdms Microparticles

### Synthesis of Poly(Dimethylsiloxane) (PDMS) Microparticles

Poly(dimethylsiloxane) (Sylgard 184) was purchased from Dow Corning. The silicone elastomer (2 mL) was mixed with 1/10 volume of a curing agent (0.2 mL). The mixture was gently stirred for 5 min, and the uncured PDMS mixture was used within an hour. The uncured PDMS mixture (2 mL) was mixed with an aqueous solution of sodium dodecyl sulfate (6 mL, 0.5 wt %) using two 10 ml Luer lock syringes. Two Luer lock syringes (Luer-LoK™) were connected through a micro-emulsion needle of gauge number 18 (Cadence Science). Each barrel of the syringes was moved to-and-fro 10 times to achieve emulsification. The emulsified mixture was directly poured into 40 mL of boiling water, and the PDMS microparticles were cured by heating the solution in a hot water bath (90° C.) for 40 min. The mixture was centrifuged for 5 min at 1500×g to remove large particles. The supernatant was centrifuged for 5 min at 8500×g to harvest the PDMS particles. The resulting pellet was dispersed in water and centrifuged again for 5 min at 8,000×g to isolate the PDMS microparticles, which was dispersed in ethanol (6 mL). The shape and size of the PDMS microparticles were evaluated using a Scanning Electron Microscope (SEM) (FEI/Aspex Explorer) by drop-casting a small amount of the PDMS microparticles dispersion in ethanol on the SEM stub and air-dried before imaging.

### Forming Pigment by Imbibing the DTE Dye in the Pre-Made PDMS Microparticles

A solution of PDMS microparticles in ethanol (1 ml) was centrifuged for 5 min at 8500×g. The microparticles pellet was dispersed in a DTE solution in chloroform (0.5 ml, 2.5 wt %). The solution was kept in dark for 1 h, then the solvent was removed using a flow of N₂. The dry residue was washed with acetonitrile (3×2 mL) and the PDMS microparticles were isolated by centrifuging the colorless solution of PDMS microparticles in acetonitrile for 5 min at 8500×g.

Forming Pigment by Doping PDMS Microparticles with DTE Dye During PDMS Synthesis

A solution of DTE in the silicone elastomer (1%) was prepared by dissolving DTE (20 mg) in silicone elastomer (2 ml), and stirring the solution at 40° C. for 20 min. The solution was cooled to room temperature, and then it was mixed with 1/10 volume of a curing agent (0.2 mL). DTE-doped microparticles were prepared according to the same method for the preparation of PDMS microparticles describe herein to provide pigments.

### Example 4

### DTE-Doped Silica Powder

A stirred solution of tetraethylorthosilicate (3.0 g, 14.4 mmol) and DTE (30 mg) in anhydrous ethanol (1.7 mL, 28.8 mmol) was treated dropwise with an aqueous HCl solution (1.4 mL, 0.1 M, 0.144 mmol) at room temperature. The mixture was irradiated with UV light (312 nm) until it became purple, and then it was stirred for 2 h at room temperature. The resultant gel was transferred into a petri dish (100×15 mm, VWR®), and was kept in an oven at 40° C. for 24 h. The resultant glass film was ground into powder using a motor and pestle. The DTE-doped silica powder was washed with ethanol (5×40 mL) before it was isolated by centrifuging for 5 min at 8500×g.

### Example 5

### Leaching Test of DTE-Doped Silica Powder

The dye-doped silica powder prepared according to the method described in Example 4 was tested for leaching in various solvents, including isopropanol, ethanol, chloroform, hexane, ethylene glycol, water and artificial sweat.

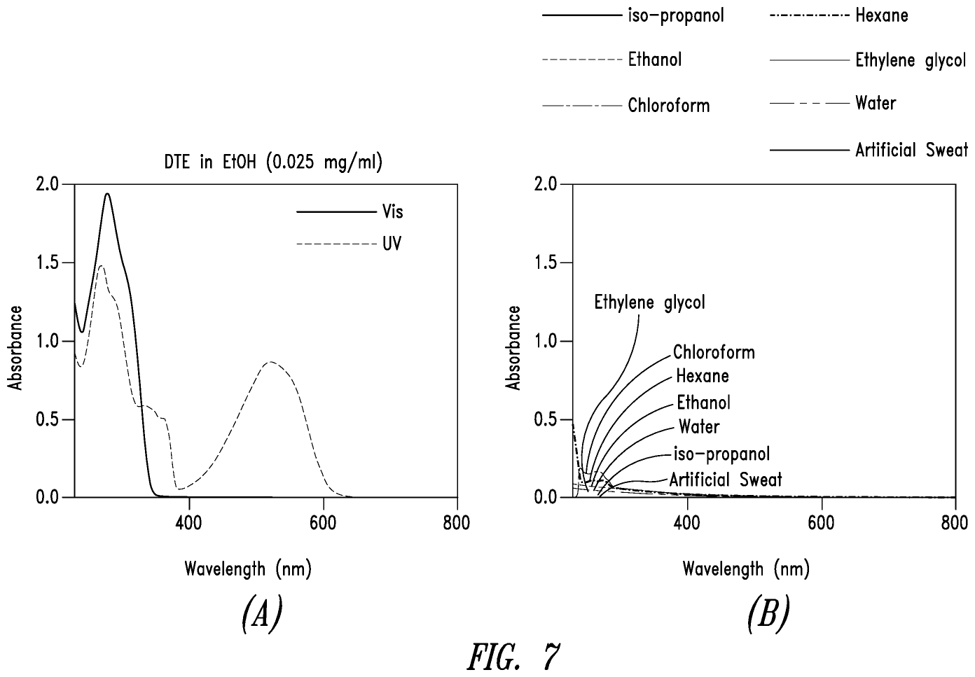

FIG. 7(A) shows the absorption spectra of DTE (0.025 mg/ml in ethanol) in ring-closed isoform (upon UV activation) and ring-opened isoform (upon visible light activation). The UV-Vis absorption was used as a reference to quantitatively determine the concentration of DTE in a solvent.

20 mg dye-loaded silica powder (1% DTE in SiO₂) was mixed with different solvents (1 ml each) for one minute followed by sonication and centrifuge. The supernatant was transferred into a cuvette and a UV-vis spectrum was acquired to determine the presence of any leaching dyes. FIG. 7(B) shows little or no absorption peaks between 400-600 nm. The test demonstrated that the DTE dye was stable and retained within the silica powder in all the solvents tested.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A UV-responsive ink formulation comprising:
a dermatologically acceptable liquid carrier; and
one or more photochromic dyes.

2. The UV-responsive ink formulation of claim 1 wherein the one or more photochromic dyes are selectively responsive to UVB radiation (290 nm-320 nm) over UVA radiation (320-400 nm).

3. The UV-responsive ink formulation of claim 1 or claim 2 wherein the one or more photochromic dyes are sensitive to one or more UV intensities corresponding to UV Indices.

4. The UV-responsive ink formulation of any one of claims 1-3 wherein the photochromic dye is a compound of spirooxazine, diarylethene, spiropyran, chromene, naphthopyran or azobenzene.

5. The UV-responsive ink formulation of any one of claims 1-4, wherein the one or more photochromic dyes are in a free form.

6. The UV-responsive ink formulation of any one of claims 1-4 wherein the photochromic dye is encapsulated in a plurality of microcapsules, each microcapsule comprising a shell enclosing a cavity, in which the photochromic dye is suspended in a liquid solvent.

7. The UV-responsive ink formulation of claim 6 wherein the shell comprises melamine-formaldehyde resin, urea-formaldehyde resins, or crosslinked gelatin.

8. The UV-responsive ink formulation of any one of claims 1-4 wherein the photochromic dye is incorporated in a plurality of solid microparticles.

9. The UV-responsive ink formulation of claim 8 wherein each solid microparticle comprises a polymer matrix, and wherein the photochromic dye is physically embedded in the polymer matrix or chemically bonded to the polymer matrix.

10. The UV-responsive ink formulation of any one of claims 8-9 wherein the polymer matrix comprises a polysiloxane.

11. The UV-responsive ink formulation of claim 10 wherein the polysiloxane is poly(dimethylsiloxane).

12. The UV-responsive ink formulation of any one of claims 8-9 wherein the polymer matrix comprises silica.

13. The UV-responsive ink formulation of any one of claims 1-4 wherein the photochromic dye is conjugated to one or more oligomers having weight-average molecular weight of less than 5000.

14. The UV-responsive ink formulation of claim 13 wherein the one or more oligomers are poly(dimethylsiloxane).

15. The UV-responsive ink formulation of claim 6-14 wherein the microcapsules, the microparticles or the oligomers have diameters in the range of 0.1-20 μm.

16. The UV-responsive ink formulation of any one of claims 1-15 wherein the one or more photochromic dyes can be represented by: 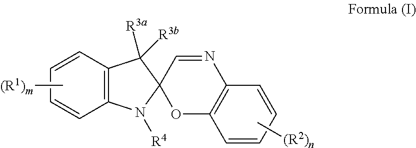
wherein,
m is 0, 1, 2, 3 or 4;
n is 0, 1, 2, 3 or 4;
R¹ at each occurrence is the same or different and independently alkyl, halo, alkoxy, haloalkyl, cyano, nitro, amino, alkenyl, alkynyl, aryl, aralkyl, heteroaryl, cycloalkyl, heterocyclyl, heteroarylalkyl, cycloalkylalkyl, or heterocyclylalkyl, or two adjacent R¹ together with the carbons to which they are attached form a carbocyclic ring;
R² at each occurrence is the same or different and independently alkyl, halo, alkoxy, haloalkyl, cyano, nitro, amino, alkenyl, alkynyl, aryl, aralkyl, heteroaryl, cycloalkyl, heterocyclyl, heteroarylalkyl, cycloalkylalkyl, or heterocyclylalkyl; or two adjacent R² together with the carbons to which they are attached form a carbocyclic ring;
each R^{3a} and R^{3b} is independently hydrogen, alkyl, or haloalkyl; and
R⁴ is alkyl, haloalkyl, aryl, heteroaryl, aralkyl, heteroarylalkyl.

17. The UV-responsive ink formulation of claim 16 wherein the photochromic dye of Formula (I) has the following isomeric structures:

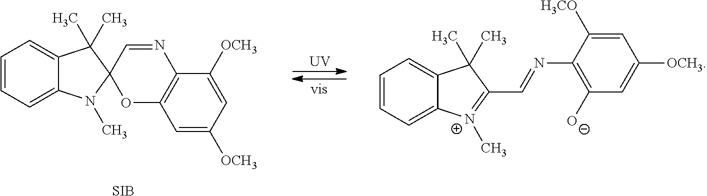 [SMILES: COc1cc(OC)c2c(c1)OC1(C=N2)N(C)c2ccccc2C1(C)C ; "UV" ; "vis" ; COc1cc([O-])c(/N=C/C2=[N+](C)c3ccccc3C2(C)C)c(OC)c1 ; "SIB"]

18. The UV-responsive ink formulation of any one of claims 1-15 wherein the one or more photochromic dyes can be represented by: 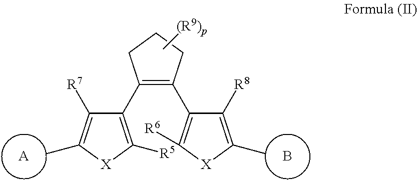
wherein,
p is 1, 2, 3, 4, 5, or 6;
A and B are the same or different and independently hydrogen, alkyl, halo, alkoxy, haloalkyl, a carbonyl-containing functional group (carboxylic acid, amide, ester, ketone, aldehyde), cyano, nitro, amino, alkenyl, alkynyl, aryl, aralkyl, heteroaryl, cycloalkyl, heterocyclyl, heteroarylalkyl, cycloalkylalkyl, or heterocyclylalkyl;
R⁵ and R⁶ are the same or different and independently alkyl, aryl or heteroaryl;
R⁷ and R⁸ are the same or different and independently hydrogen, alkyl; or
R⁷ or R⁸ connects to a respective carbon of A or B to form a benzene ring;
R⁹ is hydrogen or halogen; and
X is S or O.

19. The UV-responsive ink formulation of claim 18 wherein the photochromic dye of Formula (II) has the following isomeric structures:

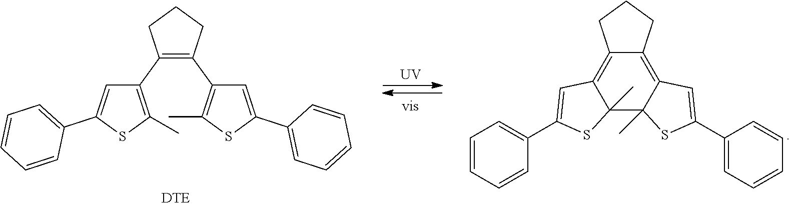

20. The UV-responsive ink formulation of any one of the preceding claims further comprising an adhesive dissolved in the dermatologically acceptable carrier.

21. The UV-responsive ink formulation of claim 20 wherein the adhesive is a film-forming agent selected from the group consisting of Shellac, nitrocellulose, hydroxymethylcellulose, hydroxyethylcellulose and zein.

22. A two-part sun care kit comprising:
a first compartment containing a sunscreen composition; and
a second compartment containing a UV-responsive ink formulation according to any one of claims 1-21.

23. The two-part sun care kit of claim 21 wherein the sunscreen composition comprises one or more mineral-based compounds.

24. The two-part sun care kit of claim 22 wherein the sunscreen composition comprises one or more photo-active chemical agents capable of absorbing UV radiation.

25. A method for managing direct UV-exposure to mammalian skin in need thereof, the method comprising:
forming an imprint of one or more photochromic dyes on the mammalian skin by applying a UV-responsive ink to the mammalian skin and allowing the UV-responsive ink to dry, and
applying a sunscreen composition on the mammalian skin and over the thin film of the one or more photochromic dyes, whereby the imprint shows a first color.

26. The method of claim 25 further comprising re-applying a sunscreen composition when the imprint changes color from the first color to a second color.

27. The method of claim 25 or claim 26 wherein the sunscreen comprises titanium oxide, zinc oxide or a combination thereof.

28. The method of claim 25 or claim 26 wherein the sunscreen comprises one or more photo-active chemical agents capable of absorbing UV radiation.

29. A multi-layer sticker comprising:
a substrate;
a dye layer overlying the substrate, wherein the dye layer comprises a broad-spectrum photochromic dye;
a filter layer overlying the dye layer, wherein the filter layer comprises one or more UV filters selectively absorbing certain UV wavelength ranges.

30. The multi-layer sticker of claim 29 wherein the UV filter is selectively absorbing UVB (290 nm-320 nm), whereby only UVA radiation can reach the dye layer.

31. The multi-layer sticker of claim 29 wherein the UV filter selectively absorbs UVA (340 nm-400 nm), whereby only UVB radiation can reach the dye layer.

32. The multi-layer sticker of claim 29 wherein the filter layer comprises a UVB filter (absorbing 290-320 nm) and a UVA1 filter (absorbing 340-400 nm), whereby only UVA2 radiation (320-340 nm) can reach the dye layer.

33. The multi-layer sticker of claim 31 wherein the UVB filter and the UVA1 filter are the same filter.

34. The multi-layer sticker of claim 31 wherein the filter layer comprises a UVB filter (absorbing 290-320 nm) and a UVA2 filter (absorbing 320-340 nm), whereby only UVA1 radiation (340 nm-400 nm) can reach the dye layer.

35. The multi-layer sticker of claim 33 wherein the UVB filter and the UVA2 filter are the same filter.

36. A method for preventing a chemical compound from transdermal delivery or minimizing systemic exposure to the chemical compound in a subject in need thereof, the method comprising:
applying a topical formulation to the subject's skin, wherein the topical formulation includes the chemical compound; a depot-forming agent; a film-forming agent and a dermatologically acceptable carrier; and
allowing the topical formulation to form a film on the subject's skin,
wherein the depot-forming agent is:
(1) a plurality of microcapsules encapsulating the chemical compound;
(2) a plurality of microparticles incorporating the chemical compound;
(3) an oligomer conjugated to the chemical compound, or
(4) the film-forming agent itself.

37. The method of claim 36 wherein the chemical compound is a photochromic dye.

38. The method of claim 36 wherein the chemical compound is an active ingredient in sunscreen.

39. The method of claim 38 wherein the active ingredient is oxybenzone or octinoxate.

40. The method of any one of claims 36-39, wherein the depot-forming agent comprises polysiloxane or silica.

41. The method of claim 40 wherein the polysiloxanes is PDMS.

42. The method of any one of claims 1-41 wherein the film-forming agent is Shellac, nitrocellulose, hydroxymethylcellulose, hydroxyethylcellulose, zein or a mixture thereof.
